(19) United States
(12) Patent Application Publication
(10) Pub. No.: US 2017/0073292 A1
(43) Pub. Date: Mar. 16, 2017

(54) SYNTHESIS OF DIKETONE COMPOUNDS FROM CARBOHYDRATES

(71) Applicants: RHODIA OPERATIONS, Paris (FR); CENTRE NATIONAL DE LA RECHERCHE SCIENTIFIQUE, PARIS Cedex 16 (FR); UNIVERSITY DE POITIERS, POITIERS Cedex (FR)

(72) Inventors: Floryan DE CAMPO, Milano (IT); Jean-Marc CLACENS, Saint-Julien-l'Ars (FR); Francois JEROME, Sèvres-Anxaumont (FR); Karine DE OLIVEIRA VIGIER, Fontaine Le Comte (FR); Fei LIU, Dalian, Liaoning (CN)

(21) Appl. No.: 15/121,823

(22) PCT Filed: Feb. 28, 2014
(86) PCT No.: PCT/CN2014/072734

§ 371 Date: Aug. 26, 2016

(51) Int. Cl. C07C 45/59; B01J 23/44; B01J 35/02; B01J 31/10; B01J 35/00; C07C 51/31; B01J 21/18

(52) U.S. Cl. CPC C07C 45/59; C07C 51/31; B01J 23/44; B01J 21/18; B01J 31/10; B01J 35/0006; B01J 35/02; B01J 2231/48; B01J 2531/002; B01J 2531/005

(57) ABSTRACT

Providing a catalytic process for preparing 1,4-diketone compounds from furanic compounds and their precursors in a liquid medium, using an acid catalytic system and optionally in the presence of hydrogen and a hydrogenation catalyst, wherein the acidic catalytic system comprises a solid acid catalyst or a mixture of water and CO₂.

### TECHNICAL FIELD

The present invention pertains to a catalytic process for converting carbohydrates to diketone compounds, and more particularly, to a catalytic process for preparing 1,4-diketone compounds from furanic compounds and their precursors.

### BACKGROUND ART

Carbohydrates, by far the largest carbon resource in nature, are recognized as a promising alternative feedstock for the production of various chemical compounds. Nevertheless, the excess oxygen content in most carbohydrates has inconvenienced their use as the starting materials in synthetic strategies. One option of circumventing this problem is to remove water from carbohydrates, so as to convert them into more attractive platform chemicals such as furan compounds, in particular 5-hydroxymethylfurfural (HMF) and its furan-class derivatives as extensively reviewed in VAN PUTTEN, ROBERT-JAN, et al. Hydroxymethylfurfural, A Versatile Platform Chemical Made from Renewable Resources. Chem. rev. 2013, vol. 113, no. 3, p. 1499-1597.

Among the numerous chemicals formed from HMF, one interesting class is 1,4-diketone, which includes important platform chemicals for producing various other compounds, such as polyols, amines, tetrahydrofuran, and lactones.

In 1991, SCHIAVO, et al. Hydrogenation Catalytique du 5-hydroxymethylfurfural en milieu aqueux. Bull. Soc. chim. Fr. 1991, vol. 128, p. 704-711. reported the conversion of HMF to a 1,4-diketone, 1-hydroxymethylhexane-2,5-dione (HMHD), by a catalytic hydrogenation reaction in an aqueous oxalic acid solution (pH=2) with a Pt/C solid catalyst. While this prior art process reportedly obtained a diketone yield of 60%, the recycling of oxalic acid is known to be problematic and poses an environmental risk.

A later-published article, VAN BEKKUM, Herman, et al. Ether Formation in the Hydrogenolysis of Hydroxymethylfurfural over Palladium Catalyst in Alcoholic Solution. Heterocycles. 2009, vol. 77, no. 2, p. 1037-1044., also mentioned the formation of HMHD from HMF hydrogenolysis, with the assistance of a Pd/C catalyst in an aqueous HCl solution. Nevertheless, this approach share the same flaw with Schiavo's work mentioned earlier: the recycling of an aqueous acid solution.

Compared to traditional routes to produce 1,4-diketones from hexoses, the above two studies used HMF as the starting material to obtain better product yield while avoiding burdensome side products (e.g. formic acid from the hexose conversion route). However, given the reactant restriction and catalyst recycling difficulty tied with the above two synthesis routes, there is still a need for an improved process to prepare 1,4-diketones without these problems or limitations.

It is therefore an object of the present invention to provide a process which not only suits for a wide range of starting materials but also leads to high diketone selectivity, with easy recycling of catalyst.

### SUMMARY OF INVENTION

The present application provides a process for preparing 1,4-diketone compounds from a furanic compound of structure (I) or a precursor thereof [hereinafter collectively referred to as Compound (F)] in a liquid medium,

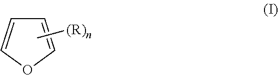

in structure (I), n is an integer between 0 and 4, and each R, being same or different, is independently selected from a group consisting of: hydrogen, —OH, —CHO, halogen, alkyl, alkenyl, alkynyl, —OR^{o}, —SR^{o}, —NHR^{o}, —NR^{o}₂, —COR^{o}, —COOR^{o}, —NH₂, —NO₂, —COOH, —CN, hydroxyalkyl, alkylcarbonyloxy, alkoxycarbonyl, alkylcarbonyl and alkylsulfonylamino, with R^{o} representing an optionally substituted alkyl, alkenyl, alkynyl, aryl, cycloalkyl, heteroaryl, or heterocycloalkyl;

and wherein the process uses at least one acidic catalytic system selected from the group consisting of:
(a) a solid acid catalyst, and
(b) a mixture of water and CO₂.

Advantageously, compared to the existing prior art, the invented process uses easily-recyclable acid catalysts and provides satisfactory product selectivity. Moreover, the catalysts used in the invented process also have a significant cost advantage per se, over the previously adopted catalysts such as oxalic acid.

Other characteristics, details and advantages of the invention will emerge even more fully upon reading the description which follows.

Throughout the description, including the claims, the term “comprising one” should be understood as being synonymous with the term “comprising at least one”, unless otherwise specified, and “between” should be understood as being inclusive of the limits.

As used herein, “alkyl” groups include saturated hydrocarbons having one or more carbon atoms, including straight-chain alkyl groups, such as methyl, ethyl, propyl, butyl, pentyl, hexyl, heptyl, octyl, nonyl, decyl, cyclic alkyl groups (or “cycloalkyl” or “alicyclic” or “carbocyclic” groups), such as cyclopropyl, cyclopentyl, cyclohexyl, cycloheptyl, and cyclooctyl, branched-chain alkyl groups, such as isopropyl, tert-butyl, sec-butyl, and isobutyl, and alkyl-substituted alkyl groups, such as alkyl-substituted cycloalkyl groups and cycloalkyl-substituted alkyl groups. The term “aliphatic group” includes organic moieties characterized by straight or branched-chains, typically having between 1 and 22 carbon atoms. In complex structures, the chains may be branched, bridged, or cross-linked. Aliphatic groups include alkyl groups, alkenyl groups, and alkynyl groups.

As used herein, “alkenyl” refers to an aliphatic hydrocarbon radical which can be straight or branched, containing at least one carbon-carbon double bond. Examples of alkenyl groups include, but are not limited to, ethenyl, propenyl, n-butenyl, i-butenyl, 3-methylbut-2-enyl, n-pentenyl, heptenyl, octenyl, decenyl, and the like.

The term “alkynyl” refers to straight or branched chain hydrocarbon groups having at least one triple carbon to carbon bond, such as ethynyl.

The term “hydroxyalkyl” refers to an alkyl group that has at least one hydrogen atom substituted with a hydroxyl group. The term “alkylcarbonyloxy” refers to a monovalent group of formula —OC(═O)-alkyl, the term “alkoxycarbonyl” refers to a group of the formula —C(═O)—O-alkyl, the term “alkylcarbonyl” refers to a group of the formula —C(═O)-alkyl, and the term “alkylsulfonylamino” refers to a group of the formula —NHS(═O)₂-alkyl.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted. The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. The term “heterocycloalkyl” refers to a cycloalkyl group in which one or more ring carbon atoms are replaced by at least one heteroatom selected from nitrogen, oxygen, and sulphur.

Notably R may comprise from 1 to 6 carbon atoms, possibly comprising at least one heteroatom selected from nitrogen, oxygen, and sulphur.

Preferably, R is selected from a group consisting of hydrogen, —CHO, alkyl, and hydroxyalkyl. In preferred embodiments, R is selected from a group consisting of hydrogen, —CHO, —CH₃ and —CH₂OH.

In particular, preferred Compound (F) may be selected from the compounds of structure (II):

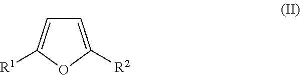

wherein R¹ and R² are defined as R above and, preferably, are independently selected from a group consisting of hydrogen, —CHO, alkyl, and hydroxyalkyl. In preferred embodiments, R¹ and R² are independently selected from a group consisting of: hydrogen, —CHO, —CH₃ and —CH₂OH.

In one preferred embodiment, the Compound (F) is 5-hydroxymethylfurfural (HMF), in which R¹ is —CHO and R² is —CH₂OH.

In another preferred embodiment, the Compound (F) is 2,5-dimethylfuran (DMF), in which R¹ and R² are both —CH₃.

In yet another preferred embodiment, the Compound (F) is 2-methyl-5-hydroxymethylfuran (MHMF), in which R¹ is —CH₃ and R² is —CH₂OH.

In yet another preferred embodiment, the Compound (F) is 2,5-dihydroxymethylfuran (DHMF), or otherwise called 2,5-furandimethanol, in which R¹ and R² are both —CH₂OH.

In yet another preferred embodiment, the Compound (F) is furfuryl alcohol (FA), in which R¹ is hydrogen and R² is —CH₂OH.

The “precursor” of the furanic compound of structure (I), as used herein, refers to any compound that is capable of being transformed into a furanic compound of structure (I) by chemical reaction, e.g. dehydration. Suitable examples of said precursor include hexoses and their derivatives including di- and polysaccharides, and are preferably selected from the group of fructose, cellulose, and inulin. Particular preferred examples of said precursor include fructose and inulin, the latter being a natural biopolymer of fructose.

The aimed 1,4-diketone products of the invented process preferably follow the structure (III) below:

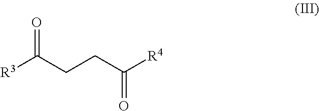

wherein R³ and R⁴ are independently selected from a group consisting of hydrogen, —OH, —CHO, halogen, alkyl, alkenyl, alkynyl, —OR^{o}, —SR^{o}, —NHR^{o}, —NR^{o}₂, —COR^{o}, —COOR^{o}, —NH₂, —NO₂, —COOH, —CN, hydroxyalkyl, alkylcarbonyloxy, alkoxycarbonyl, alkylcarbonyl and alkylsulfonylamino, wherein R^{o} is as above defined. Preferably, R³ and R⁴ are independently selected from hydrogen, —OH, —OR^{o}, and alkyl.

Preferred 1,4-diketone compounds of formula (III) are notably selected from 1-hydroxymethylhexane-2,5-dione (HMHD), levulinic acid (LA), and 2,5-hexanedione (HDX).

According to a preferred embodiment, the invented process comprises reacting the Compound (F) in the presence of hydrogen and at least one hydrogenation catalyst [Catalyst (H)], wherein the Catalyst (H) may comprise at least one metal [Metal (M)] selected from the group consisting of Pd, Ru, Pt, Rh, Ir, Fe, Co, Ni, Cu, Ag, Re, Os, and Au.

When the invented process is carried out in the presence of hydrogen, such may be directly introduced in gaseous form or produced by at least one hydrogen generating compound (such as ammonia borane) present in the liquid medium. Preferably, the Catalyst (H) is a supported hydrogenation catalyst, i.e. further comprising a support material on which Metal (M) is deposited. The selection of said support material is not strictly limited, and preference is given to using activated carbon, silicon carbide, aluminum oxide, silicon dioxide, titanium dioxide, zirconium dioxide, magnesium oxide, zinc oxide or mixtures thereof, more preferably activated carbon.

In use, the Catalyst (H) may be a supported hydrogenation catalyst comprising at least one Metal (M) selected from the group consisting of Pd, Ru, Pt, Rh, Ir, Fe, Co, Ni, Cu, Ag, Re, Os, Au, and any combinations thereof. The loading of Metal (M) can vary within a large range, e.g., from 0.1-10 wt % with respect to the weight of the support. However, for noble metals such as Ru, Ph, Pd, Pt, Ir, etc., the metal loading is preferably about 0.1 to about 5 wt %, and more preferably about 0.1 to about 1 wt % with respect to the weight of the support.

In one preferred embodiment, the Metal (M) is Pd.

Preferred examples of Catalyst (H) are selected from the group consisting of Pd/C, Pearlman's catalyst, Adam's catalyst, Pt/C, and Raney-Ni, with Pd/C being particularly preferred.

If used, the loading of Catalyst (H) is generally from 1 to 20% by weight, preferably from 2 to 15% by weight, and more preferably from 5 to 10% by weight, with respect to the weight of Compound (F).

After use, the Catalyst (H) can be easily recovered (e.g. by filtration) and re-used without further purification. Advantageously, the Catalyst (H) can be successfully recovered after simple filtration and re-used for at least three times without noticeable decrease of the reaction yield.

For the purpose of the present invention, the term “liquid medium” refers to a medium that is predominantly a liquid under the reaction condition of the process invention, and encompasses solutions, dispersions, emulsions, and the like. As used herein, the term “liquid medium” can indicate a pure liquid or a combination of two or more liquids.

According to the process invention, the liquid medium may comprise water or a non-aqueous liquid. Examples of said non-aqueous liquid may be selected from the group of: 2-Methyl-tetrahydrofuran (2-MeTHF), methylisobutylketone, toluene, diethylether, dioxane, tetrahydrofuran (THF), and a combination thereof. In the preferred embodiments, the liquid medium contains THF, water, or a mixture thereof.

The reaction temperature for the process may be generally comprised between 50 and 200° C., and reaction time for said process is generally comprised between 1 and 30 hours.

Regarding the Process Using an Acidic Catalytic System Comprising a Solid Acid Catalyst

As aforementioned, the acidic catalytic system used in the invented process may comprise a solid acid catalyst. Non-limited examples of the applicable solid acid catalyst include acid ion exchange resins, zeolites, sulfated zirconia, zirconia, sulfated titania, tungsted zirconia, boron phosphate, and acidic clays such as, in particular, smectites (e.g. montmorillonites, beidellites, nontronites, hectorites, stevensdites and saponites).

For the purpose of the invention, the term “acid ion exchange resin” refers to a cation exchange resin in the hydrogen form wherein the hydrogen ions are bound to the active sites which can be removed either by dissociation in solution or by replacement with other positive ions.

Representative of acid ion-exchange resins are strong-acid ion exchangers, such as those resins or polymers having a plurality of pendant sulfonic acid groups. Examples include sulphonated polystyrene or poly(styrene-divinylbenzene) copolymer and sulphonated phenol-formaldehyde resins. The sulphonated resins are commercially available in water swollen form as gellular, micro-recticular and macro-recticular types. Specific examples of suitable resins are Amberlite® IR-120H, Amberlyst® 15, Amberlyst® 31 and 131 Dowex® 50-X-4, Dowex® MSC-1H, Duolite® c-26, Permutit® QH, Chempro® C-2, Purolite® CT-124, Bayer K-1221 and Imac® C8P/H, as well as the resins marketed under the trademark Nafion®.

Other examples of solid acid catalysts include ZSM-5 zeolite catalyst.

Preferred Compounds (F) for such a process include HMF, fructose, and inulin.

The desired loading of said solid acid catalyst is generally from 5 to 30% by weight, preferably from 10 to 30% by weight, and more preferably from 15 to 25% by weight, based on the weight of Compound (F).

The reaction temperature for the process can be advantageously set in a mild condition, generally between 50 and 100° C., and preferably between 70 and 90° C.

Reaction time for said process is generally between 1 and 30 hours, preferably between 5 and 20 hours, more preferably between 10 and 20 hours.

In a specific embodiment, the liquid medium for the process comprises THF, or a THF/water mixture.

It is in principle possible to use all reactors which are basically suitable for gas/liquid reactions at the given temperature and the given pressure for the catalytic process of the invention.

Preferably, the process using an acidic catalytic system comprising a solid acid catalyst is carried out in the presence of hydrogen and a Catalyst (H). The hydrogen pressure is usually adjusted in a range of 10 to 100 bar, preferably between 30 and 80 bar, and more preferably between 40 and 60 bar.

According to certain embodiments, the Compound (F) is mixed and heated in the liquid medium within a reactor, in the presence of the Catalyst (H) and a solid acid catalyst, in the presence of hydrogen.

The introduction of the Compound (F), the liquid medium, the Catalyst (H), and the solid acid catalyst into said reactor can be carried out simultaneously or separately and/or sequentially. The reaction can be carried out continuously, in the semibatch mode, in the batch mode, admixed in product as solvent or without admixing in a single pass.

The reaction mixture formed in the reaction generally comprises the target 1,4-diketone compound, the Catalyst (H), the solid acid catalyst, possibly unreacted reactant(s) and possibly present byproduct(s) formed from the reaction.

Any excess reactant(s) present, any liquid medium present, the Catalyst (H), the solid acid catalyst, and the by-product present can be removed from the reaction mixture, typically according to standard separation techniques. The 1,4-diketone product obtained can be worked up further.

Notably, the solid acid catalyst may be recovered together with the Catalyst (H), such as by filtration, and re-used with or without further purification. Advantageously, the solid acid catalyst can be successfully recovered after simple filtration and re-used without noticeable decrease of the reaction yield.

Regarding the Process Using an Acidic Catalytic System Comprising a Mixture of Water and CO₂

Alternatively, the acidic catalytic system used in the invented process may comprise a mixture of water and CO₂ in place of the aforementioned solid acid catalyst.

Besides the obvious catalyst cost advantage, removing CO₂ and water from the aimed diketone products is notably easy and convenient. Practically, the gaseous component CO₂ of this acidic catalytic system can be simply vented from the reactor upon reaction completion, together with un-reacted hydrogen, if present.

Advantageously, the liquid medium in such a process can use water as the sole liquid component for easy recycling or, alternatively, comprises a mixture of water and a non-aqueous liquid with varied proportion. Selection of said non-aqueous liquid is not particularly limited, as long as it forms an azeotrope with water and preferably water-miscible. Examples of said non-aqueous liquid include 2-MeTHF, methylisobutylketone, toluene, diethylether, dioxane, and THF, of which THF is preferred.

Surprisingly, as noted by the Applicant, the 1,4-diketone product selectivity of such a process can be conveniently tuned by changing the liquid composition of the liquid medium.

Preferred Compounds (F) for such a process include HMF, DMF, FA, MHMF, DHMF, fructose, and inulin.

Optionally, the process using an acidic catalytic system comprising a mixture of CO₂ and water is carried out in the presence of hydrogen and a Catalyst (H).

When the process is carried out in the presence of hydrogen, hydrogen pressure is generally between 0.5 and 15 bar, and preferably between 0.5 and 10 bar.

In general, a total pressure of hydrogen and CO₂ present in the reaction system is between 20 to 60 bar, preferably between 30 and 50 bar.

Notably, the process using an acidic catalytic system comprising a mixture of CO₂ and water can obtain a high 1,4-diketone product selectivity in the absence of hydrogen and Catalyst (H). This is evident in certain especially preferred embodiments (e.g. when DMF or FA is used as Compound (F)).

The reaction temperature is usually set between 80 and 200° C., and preferably between 100 and 130° C.

Reaction time for said process is generally between 1 and 30 hours, preferably between 5 and 20 hours, more preferably between 10 and 20 hours.

To carry out the reaction, typically, the Compound (F) is mixed and heated in an aqueous medium within a reactor, in the presence of CO₂ and optionally in the presence of hydrogen and the hydrogenation Catalyst (H). In a preferred embodiment, CO₂ is progressively introduced throughout the reaction.

The reaction can be carried out continuously, in the semibatch mode, in the batch mode, admixed in product as solvent or without admixing in a single pass.

It is in principle possible to use all reactors which are basically suitable for gas/liquid reactions at the given temperature and the given pressure for the catalytic process of the invention.

The reaction output formed in the reaction generally comprises the aimed products of 1,4-diketone compound, CO₂, possibly unreacted Compound (F), possibly present hydrogen and Catalyst (H), and possibly present co-product formed from the reaction.

CO₂ and hydrogen (if present) can be vented from the reactor to the atmosphere, and the Catalyst (H), if present, can be recycled by any liquid-solid separation approach (e.g. filtration). The 1,4-diketone product obtained can be worked up further.

### DESCRIPTION OF EMBODIMENTS

The following examples are provided to illustrate preferred embodiments of the invention and are not intended to restrict the scope thereof.

### EXAMPLES

### Example 1 Preparation of HMHD from HMF Using a Solid Acid Catalyst in the Presence of Hydrogen and a Catalyst (H)

To a 5 mL THF/H₂O (9:1) mixture containing 9.75 mg of Pd/C and 16.5 mg of Amberlyst® 15 (hereinafter abbreviated as “A15”), HMF (150 mg) was added. The thus obtained mixture was then placed inside a 45 ml autoclave and flushed with hydrogen. Subsequently, the autoclave was heated to 80° C. under a hydrogen pressure of 50 bar, for 15 hours. The reaction mixture was then let cool to room temperature, after which the reactor was vented and opened. A syringe filter was used to remove the solid catalysts from the reaction mixture, and the remaining liquid was analysed by GC using biphenyl as the internal standard. The HMF conversion was measured to be 100%, and the yield of HMHD was 77%.

The major co-product was LA, another 1,4-diketon compound, with 10% yield. Total carbon mass balance of this reaction reached 84%.

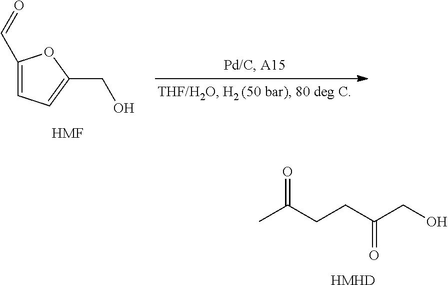

### Example 2 Preparation of HMHD from Fructose Using a Solid Acid Catalyst in the Presence of Hydrogen and a Catalyst (H)

To a 5 ml THF/H₂O (9:1) mixture was added 250 mg Fructose, 16.25 mg Pd/C and 27.5 mg of A15 catalyst. The thus obtained mixture was then placed inside a 45 ml autoclave and flushed with hydrogen. Subsequently, the autoclave was heated to 80° C. under a hydrogen pressure of 20 bar, for 20 hours. The reaction mixture was then let cool to room temperature, after which the reactor was vented and opened. A syringe filter was used to remove the solid catalysts from the reaction mixture, and the remaining liquid was analysed by GC using biphenyl as the internal standard. The fructose conversation was measured to be 95%, and the yield of HMHD was 55%. The main co-products were LA and HMF, with 11% and 12% yield respectively. Total carbon mass balance of this reaction reached 82%.

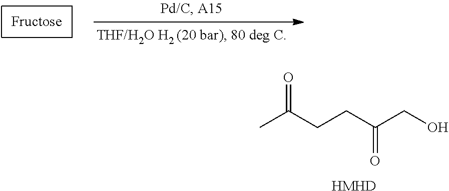

### Example 3 Preparation of HDX from DMF Using CO₂/H₂O Catalyst

A 5 ml water solution of DMF (150 mg, 1.56 mmol) was placed inside an autoclave and CO₂ was introduced, to reach a pressure of 40 bar. Under this pressure, the reaction mixture was stirred and heated to 150° C., for 15 hours. The reaction mixture was then let cool to room temperature, after which the reactor was vented and opened to release CO₂. The thus obtained aqueous mixture was analysed by GC using biphenyl as the internal standard. The DMF conversion was 100%, and the yield of HDX was as high as 95%.

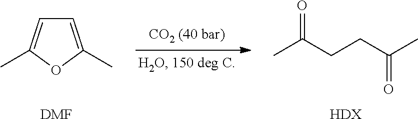

### Example 4 Preparation of LA from FA Using CO₂/H₂O Catalyst

A 5 mL water solution of FA (150 mg, 1.56 mmol) was placed inside an autoclave and CO₂ was introduced, to reach a pressure of 40 bar. Under this pressure, the reaction mixture was stirred and heated to 150° C., for 15 hours. The reaction mixture was then let cool to room temperature, after which the reactor was vented and opened to release CO₂. The thus obtained aqueous mixture was analysed by GC using biphenyl as the internal standard. The FA conversation was higher than 95%, and the yield of LA was 55%.

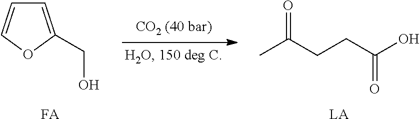

### Example 5 Preparation of HMHD from DHMF Using CO₂/H₂O Catalyst in the Presence of Hydrogen and Catalyst (H)

To a mixture of deionized water (5 ml) and DHMF (150 mg, 1.17 mmol) was added Pd/C catalyst (3 mg, 1.4 μmol). The resulting composition was then placed inside an autoclave and was flushed with hydrogen, until reaching a hydrogen pressure of 1 bar. Subsequently, CO₂ was introduced up to a pressure of 39 bar (i.e. a total gas pressure of 40 bar). Under this gas pressure, the reaction mixture was stirred and heated to 120° C. for 10 hours. The reaction mixture was then let cool to room temperature, after which the reactor was vented and opened to release CO₂ and hydrogen. A syringe filter was used to remove the solid Pd/C catalyst from the reaction mixture, and the remaining aqueous composition was analysed by GC using biphenyl as the internal standard. The DHMF conversion exceeded 95%, and the yield of HMHD was 60%.

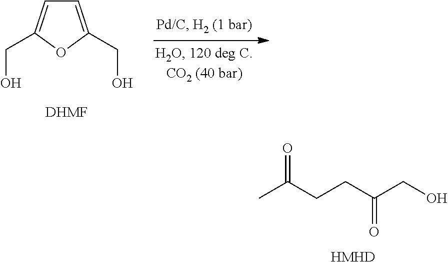

### Example 6 Preparation of HMHD from HMF Using CO₂/H₂O Catalyst in the Presence of Hydrogen and Catalyst (H)

To a mixture of deionized water (5 ml) and HMF (150 mg, 1.19 mmol) was added Pd/C catalyst (11 mg, 5.2 μmol). The resulting composition was then placed inside an autoclave and was flushed with hydrogen, until reaching a hydrogen pressure of 10 bar. Subsequently, CO₂ was introduced up to a pressure of 30 bar (i.e. a total gas pressure of 40 bar). Under this gas pressure, the reaction mixture was stirred and heated to 120° C. for 15 hours. The reaction mixture was then let cool to room temperature, after which the reactor was vented and opened to release CO₂ and hydrogen. A syringe filter was used to remove the solid Pd/C catalyst from the reaction mixture, and the remaining aqueous composition was analysed by GC using biphenyl as the internal standard. The DHMF conversion was near 100%, and the yield of HMHD was 70%.

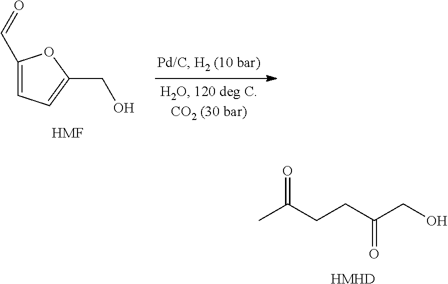

### Example 7 Preparation of HMHD from Inulin Using CO₂/H₂O Catalyst

A 5 ml water solution of inulin (150 mg, 3 wt %) was placed inside an autoclave and CO₂ was introduced, to reach a pressure of 40 bar. Under this pressure, the reaction mixture was stirred and heated to 150° C., for 15 hours. The reaction mixture was then let cool to room temperature, after which the autoclave reactor was vented and opened to release CO₂. The thus obtained aqueous mixture was analysed by GC using biphenyl as the internal standard. The conversion of inulin was near 100%, and the overall yield of HMHD from inulin was about 15%.

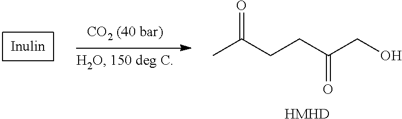

### Example 8 Preparation of HMHD from Fructose Using CO₂/H₂O Catalyst

A 5 ml water solution of fructose (150 mg, 3 wt %) was placed inside an autoclave and CO₂ was introduced, to reach a pressure of 40 bar. Under this pressure, the reaction mixture was stirred and heated to 150° C., for 15 hours. The reaction mixture was then let cool to room temperature, after which the autoclave reactor was vented and opened to release CO₂. The thus obtained aqueous mixture was analysed by GC using biphenyl as the internal standard. The conversion of fructose was near 100%, and the overall yield of HMHD from fructose was about 36%.

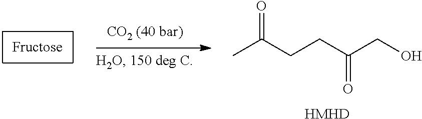

### Example 9 Preparation of HMHD from Inulin Using a Solid Acid Catalyst in the Presence of Hydrogen and a Catalyst (H)

To a 5 ml THF/H₂O (9:1) mixture was added 250 mg Inulin, 16.25 mg Pd/C and 27.5 mg of A15 catalyst. The thus obtained mixture was then placed inside a 45 ml autoclave and flushed with hydrogen. Subsequently, the autoclave reactor was heated to 80° C. under a hydrogen pressure of 20 bar, for 20 hours. The reaction mixture was then let cool to room temperature, after which the reactor was vented and opened. A syringe filter was used to remove the solid catalysts from the reaction mixture, and the remaining liquid was analysed by GC using biphenyl as the internal standard. The inulin conversation reached 95%, and the yield of HMHD was 36%.

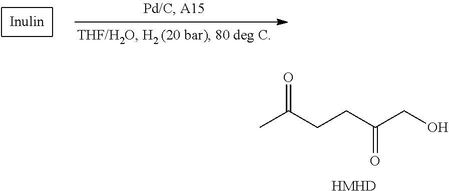

1. A process for preparing 1,4-diketone compounds from a furanic compound of structure (I) or the precursor thereof [Compound (F)] in a liquid medium, 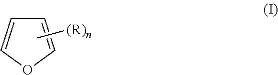
wherein:
in structure (I), n is an integer between 0 and 4, and each R, being same or different, is independently selected from a group consisting of: hydrogen, —OH, —CHO, halogen, alkyl, alkenyl, alkynyl, —OR^{o}, —SR^{o}, —NHR^{o}, —NR^{o}₂, —COR^{o}, —COOR^{o}, —NH₂, —NO₂, —COOH, —CN, hydroxyalkyl, alkylcarbonyloxy, alkoxycarbonyl, alkylcarbonyl and alkylsulfonylamino, with R^{o} representing an optionally substituted alkyl, alkenyl, alkynyl, aryl, cycloalkyl, heteroaryl, or heterocycloalkyl;
and wherein the process uses at least one acidic catalytic system selected from the group consisting of:
(a) a solid acid catalyst, and
(b) a mixture of water and CO₂.

2. The process of claim 1, wherein the Compound (F) is selected from the compounds of structure (II): 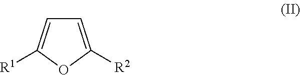
wherein R¹ and R² are independently selected from a group consisting of: hydrogen, —OH, —CHO, halogen, alkyl, alkenyl, alkynyl, —OR^{o}, —SR^{o}, —NHR^{o}, —NR^{o}₂, —COR^{o}, —COOR^{o}, —NH₂, —NO₂, —COOH, —CN, hydroxyalkyl, alkylcarbonyloxy, alkoxycarbonyl, alkylcarbonyl and alkylsulfonylamino.

3. The process of claim 1, wherein the Compound (F) is selected from the group consisting of: 5-hydroxymethylfurfural (HMF), 2-methyl-5-hydroxymethylfuran (MHMF), 2,5-dimethylfuran (DMF), 2,5-dihydroxymethylfuran (DHMF), and furfuryl alcohol (FA).

4. The process of claim 1, wherein the precursor of Compound (F) is selected from fructose and inulin.

5. The process of claim 1, wherein the 1,4-diketone compounds are those following the structure (III) below: 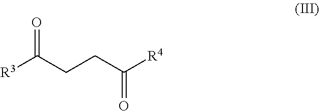
wherein R³ and R⁴ are independently selected from a group consisting of hydrogen, —OH, —CHO, halogen, alkyl, alkenyl, alkynyl, —OR^{o}, —SR^{o}, —NHR^{o}, —NR^{o}₂, —COR^{o}, —COOR^{o}, —NH₂, —NO₂, —COOH, —CN, hydroxyalkyl, alkylcarbonyloxy, alkoxycarbonyl, alkylcarbonyl and alkylsulfonylamino.

6. The process of claim 5, wherein the 1,4-diketone compounds are selected from 1-hydroxymethylhexane-2,5-dione (HMHD), levulinic acid (LA), and 2,5-hexanedione (HDX).

7. The process of claim 1, wherein the process comprises reacting the Compound (F) in the presence of hydrogen and at least one hydrogenation catalyst [Catalyst (H)], wherein the Catalyst (H) comprises at least one metal [Metal (M)] selected from the group consisting of Pd, Ru, Pt, Rh, Ir, Fe, Co, Ni, Cu, Ag, Re, Os, and Au.

8. The process of claim 7, wherein the Catalyst (H) is a supported catalyst, which further comprises a support material on which the Metal (M) is deposited, wherein the support material is selected from a group consisting of activated carbon, silicon carbide, aluminum oxide, silicon dioxide, titanium dioxide, zirconium dioxide, magnesium oxide, zinc oxide and mixtures thereof.

9. The process of claim 7, wherein the Catalyst (H) is selected from the group consisting of Pd/C, Pearlman's catalyst, Adam's catalyst, Pt/C, and Raney-Ni.

10. The process of claim 1, wherein the acidic catalytic system comprises a solid acid catalyst.

11. The process of claim 10, wherein the solid acid catalyst is selected from a group consisting of acid ion exchange resins, zeolites, sulfated zirconia, zirconia, sulfated titania, tungsted zirconia, boron phosphate, and acidic clays.

12. The process of claim 10, wherein the solid acid catalyst is an acid ion exchange resin selected from a group consisting of sulphonated polystyrene or poly(styrene-divinylbenzene) copolymer and sulphonated phenol-formaldehyde resins.

13. The process of claim 10, wherein the solid acid catalyst is a ZSM-5 zeolite catalyst.

14. The process of claim 10, wherein the process is carried out in the presence of hydrogen and a Catalyst (H).

15. The process of claim 1, wherein the acidic catalytic system comprises a mixture of water and CO₂.

16. The process of claim 15, wherein the liquid medium uses water as the sole liquid component.

17. The process of claim 15, wherein the liquid medium comprises a mixture of water and a non-aqueous liquid.

18. The process of claim 15, wherein the process is carried out in the presence of hydrogen and a Catalyst (H).

19. The process of claim 15, wherein the process is carried out in the absence of hydrogen and a Catalyst (H).

20. The process of claim 15, wherein the Compound (F) is selected from the group consisting of HMF, DMF, FA, MHMF, DHMF, fructose, and inulin.
